(19) United States
(12) Patent Application Publication
(10) Pub. No.: US 2010/0234325 A1
(43) Pub. Date: Sep. 16, 2010

(54) 4-Peroxycyclophosphamide use thereof

(75) Inventor: Robert Frederick Struck, Birmingham, AL (US)

Correspondence Address: Robert F. Struck, 3533 Laurel View Lane, Birmingham, AL, 35216, US

(73) Assignee: Robert Frederick Struck, Birmingham, AL (US)

(21) Appl. No.: 12/381,430

(22) Filed: Mar. 13, 2009

(51) Int. Cl. C07F 9/547; A61K 31/675; A61P 35/04

(52) U.S. Cl. 514/110; 568/12

(57) ABSTRACT

4-Peroxycyclophosphamide is provided along with its method of preparation. The compound is useful for treating human cancer, particularly human primary and metastatic malignant brain tumors.

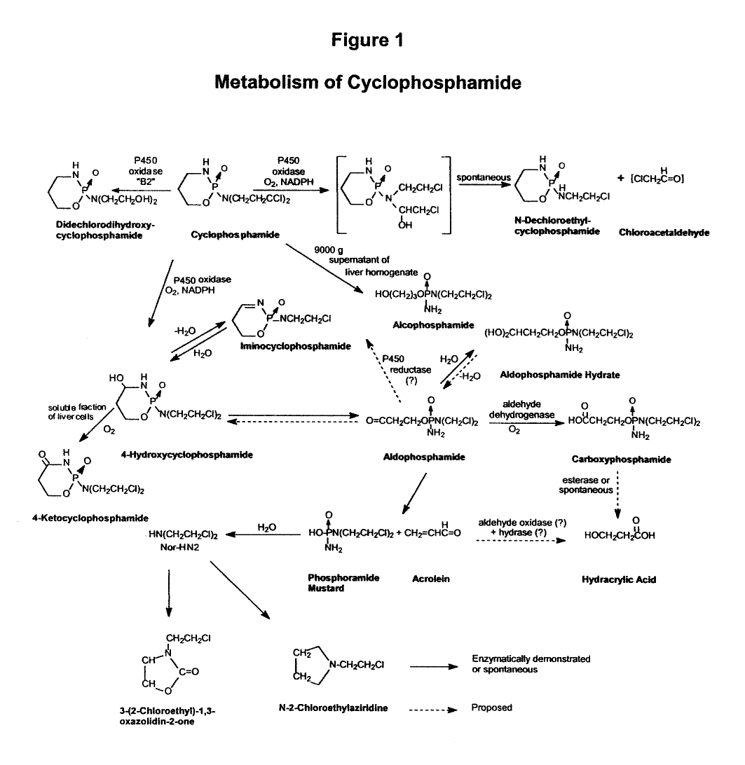

### TECHNICAL FIELD

The present invention relates to the single compound, 4-peroxy-cyclophosphamide (also referred to herein as peroxyCPA), which exhibits anticancer activity against human cancers. Accordingly, the present invention also relates to pharmaceutical compositions comprising this particular compound, as well as a method of using it in treating human cancer, particularly primary and metastatic, malignant brain tumors. No composition of matter is claimed for peroxyCPA, 4-hydroperoxycyclophosphamide (4-HC), 4-hydroperoxyifosfamide, 4-hydroxycyclophosphamide, 4-hydroxyifosfamide, phosphoramide mustard (PM) or isophosphoramide mustard (IPM, palifosfamide, Zymafos®).
PeroxyCPA

C₁₄H₂₈Cl₄N₄O₆P₂

### BACKGROUND OF INVENTION

Primary brain tumors are among the most difficult tumors to treat and constitute the second leading cause of death from cancer in children, resulting in 15,000 deaths annually in children and adults in the U.S. The major types of primary brain tumors are glioblastoma multiforme, anaplastic astrocytoma, anaplastic oligodendroglioma, ependymoma, brainstem glioma, spinal cord tumor, medulloblastoma and germ cell tumors, with treatment typically consisting of surgery, radiotherapy and chemotherapy (1).

Many drugs have been used to treat primary brain tumors, the major class being the nitrosoureas, particularly carmustine (BCNU) and lomustine (CCNU). The most widely used drug combination has been PCV (procarbazine, lomustine, vincristine), but many other drugs used to treat other tumor types have been employed for brain tumors also and include the following: ACNU, cisplatin, carboplatin, tamoxifen, irinotecan, temozolomide, 6-thioguanine, dibromodulcitol, thiotepa, cyclophosphamide (CPA), prednisone, etoposide, vinblastine and bleomycin (1, 2).

Treatment of metastases particularly of primary tumors of the breast and lung is also a challenge; 25% of all cancer patients develop brain metastases, the most common primary tumors being, in addition to lung and breast, melanoma, colon and kidney in adults and sarcoma and germ cell tumors in children. Major drugs used for metastatic brain tumors are CPA, 5-fluorouracil, methotrexate, doxorubicin, prednisone, cisplatin and etoposide. Whereas spinal metastases are treated with radiation but not chemotherapy, neoplastic meningitis, a common occurrence in patients with solid tumors, lymphomas and leukemias, is treated with both, and several established drugs such as methotrexate, ara-C and thiotepa, as well as some newer agents such as two CPA analogs related to peroxyCPA, i.e., mafosfamide and 4-hydroperoxycyclophosphamide (4-HC), have also been investigated in this disease (1).

While CPA has been used to treat both primary brain tumors and other tumors that have metastasized to the brain, it suffers from a requirement to be metabolically activated in the liver to 4-hydroxycyclophosphamide (HO-CPA), a non-alkylating metabolite at physiological pH (3), which is converted in vivo to the ultimate alkylating metabolite, phosphoramide mustard (PM), and can be transported via circulation to tumor cells in the brain. However, both of these potentially therapeutic metabolites, particularly PM, are polar molecules not likely readily transported through the blood brain barrier into brain tumor cells, although the clinical activity of CPA vs. both primary brain tumors and metastatic cancer in the brain clearly indicates some transport of one or both into the brain.

Several years ago, Benckhuijsen (4) prepared peroxyCPA and 4-HC by Fenton oxidation of CPA, and subsequent investigations led to evaluation of the activity of peroxyCPA against intraperitoneally-implanted L1210 leukemia in vivo in which curative activity was observed (5). Later studies led to evaluation against intracerebrally implanted L1210 leukemia in order to compare its activity and that of 4-HC with CPA in this experimental model. While 4-HC was essentially inactive against an implant of 10⁴ cells, peroxyCPA at its highest non-toxic dose of 300 mg/kg reduced the leukemia cell burden to one cell (1.0×10⁰) and yielded an increase in lifespan (ILS) of 85% over controls. Lower doses produced a normal dose response with greater numbers of leukemia cells surviving therapy. In contrast, CPA at its highest non-toxic dose reduced the leukemia cell burden to 1.3×10² cells, a very significant two-log cell kill less than that produced by peroxyCPA. Against an implant of 10⁵ L1210 cells, peroxyCPA similarly reduced the leukemia cell burden by two orders of magnitude more than that produced by CPA, giving an ILS of 100% compared to 37% for CPA at their highest non-toxic doses. In this model, the highest non-toxic dose of CPA is typically 250-300 mg/kg (6).

The significant results of these studies is the superior activity of peroxyCPA compared to CPA, a drug with some utility against primary brain tumors in patients and with an important role particularly against clinical breast and lung cancer that has metastasized to the brain (1). It seems reasonable to propose that the superiority of peroxyCPA in this model, compared to 4-HC and CPA, resulted from the greater lipophilicity of peroxyCPA, which permitted its greater transport through the blood brain barrier (BBB) and greater availability for exerting an antileukemic effect in the brains of experimental animals upon systemic administration, consistent with recently determined C log P values of 2.39, 1.19, 0.65 and −0.42 for peroxyCPA, 4-HC, 4-hydroxyCPA and phosphoramide mustard, resp. It is noted that 4-HC was active against D54 and TE-671 brain tumor models upon intrathecal administration (7) and against D54 tumor upon intraarterial or intravenous administration (8) leading the authors to propose intraarterial administration of 4-HC for clinical treatment of brain tumors (8).

Direct evidence of the activity of peroxyCPA vs. brain tumor cells in vitro and in vivo was shown by recent studies that demonstrated superior activity of peroxyCPA in vitro vs. three glioma cell lines (SF-268, SNB-7, SNB-19) compared to the clinical drugs BCNU and temozolomide. Additional in vitro studies vs. U251, D54 and MDA-MB-361 cells also included 4-HC. Activity in vivo vs. intracerebrally (IC)-implanted human glioblastoma xenograft U-251, yielded a 300-350% ILS for peroxyCPA vs. 110-150% for BCNU in one study, and a second yielded typical activity for BCNU (130% ILS), concomitant with 180% ILS for peroxyCPA consistent with a possible role for treatment of neurological cancers, since BCNU is both the experimental and clinical standard for activity vs. primary brain tumors. A potential major advantage of peroxyCPA as a possible future clinical drug for treatment of brain tumors (or other tumor types) compared to the nitrosoureas BCNU and CCNU is the likely absence of delayed toxicity typically accompanying use of the nitrosoureas.

The antitumor activity of nitrogen mustard and CPA correlates with their ability to produce DNA-DNA crosslinks in tumor cells (9). These agents crosslink tumor cell DNA in the major groove via a 5′→3′, N⁷-guanine-N⁷-guanine crosslink in a G-X-C sequence, as shown by Loechler (10) and Hopkins (11) for nitrogen mustard and by Povirk (12) and Dong et al. (13) for PM. Similar studies by Struck et al. revealed that isophosphoramide mustard (IPM), the active metabolite of ifosfamide, which produces a 7-atom crosslink in DNA instead of a 5-atom crosslink like nitrogen mustard and CPA, similarly preferentially crosslinks a G-X-C sequence (14). Evidence that peroxyCPA similarly crosslinks DNA was shown in an experiment using pBR322 DNA.

PeroxyCPA (NSC 176986) was compared in the same experiment with CPA (NSC 26271) and ifosfamide (NSC 109724) against IC-implanted MX-1 human breast tumor, using BCNU (NSC 409962) as a control. The results indicate superior activity for peroxyCPA compared to BCNU, peroxyCPA producing a 69% ILS vs. a 13% ILS for BCNU. Consequently, peroxyCPA could yield possible future benefit to breast cancer patients presenting with brain metastases, possibly identifying a relatively major role for peroxyCPA for this condition, in addition to primary brain tumors.

The stability of peroxyCPA in human serum was determined using ³¹P-NMR analysis. PeroxyCPA (ppm 12.40) demonstrated good stability with 74% remaining after 6 hours but was accompanied by slow conversion to 4-HC (14.20 ppm) at ambient temperature. During this time period, no hydroxyCPA (10.80 ppm) or PM (17.40 ppm) was observed. This result is consistent with conversion of 4-HC to hydroxyCPA in vivo being controlled primarily by cellular peroxidases as proposed by Hilton (John Hilton, Johns Hopkins Oncology Center) and secondarily by direct hydrolysis in circulation.

### SUMMARY OF INVENTION

The present invention relates to peroxyCPA which possesses anticancer activity.

The compound of the present invention is represented by the following formula:

C₁₄H₂₈Cl₄N₄O₆P₂

PeroxyCPA I

Pharmaceutically acceptable salts of compounds of formula I can also be provided.

Still other objects and advantages of the present invention will become readily apparent by those skilled in the art from the following detailed description, wherein it is shown and described only the preferred embodiments of the invention, simply by way of illustration of the best mode contemplated of carrying out the invention. As will be realized, the invention is capable of modifications in various obvious respects, without departing from the invention. Accordingly, the description is to be regarded as illustrative in nature and not as restrictive.

### BEST AND VARIOUS MODES FOR CARRYING OUT INVENTION

The present invention is concerned with a CPA derivative represented by the following formula:

C₁₄H₂₈Cl₄N₄O₆P₂

PeroxyCPA I

Pharmaceutically acceptable salts of compounds of formula I can be those derived from mineral and organic acids.

It has been found that the compound of formula I is advantageously useful in treating mammalian cancer, especially human cancer. This compound has been shown to exhibit generally superior activity in comparison to CPA against human primary brain tumors and against human breast tumors in the brain.

### EXAMPLE 1

PeroxyCPA is conveniently prepared by Fenton oxidation of CPA. CPA and ferrous sulfate in aqueous solution at 0° C. are treated dropwise with 30% hydrogen peroxide while maintaining the temperature of the reaction solution below 10° C. Extraction with chloroform yields an extract containing peroxyCPA, 4-HC—, 4-ketocyclophosphamide and CPA. Concentration of the extract in vacuo and filtration removes most of the 4-ketocyclophosphamide. The filtrate is washed with N potassium hydroxide solution to convert 4-HC to peroxyCPA, separated, dried over sodium sulfate, filtered and evaporated to dryness. The residue is separated by preparative thin layer chromatography (TLC) on silica gel (R_{f} 0.8) or silica gel column chromatography in acetone:chloroform 3:1 (v/v) to isolate peroxyCPA by elution from the TLC band and evaporation of the eluate or by evaporation of the column eluate containing peroxyCPA. The residue is crystallized from ether:acetone and characterized by elemental, mass spectral and nuclear magnetic resonance (NMR) analysis (5).

A high performance liquid chromatographic (HPLC) method was devised for peroxyCPA.

### REFERENCES

1. Bast, R. C., Kufe, D. W., Pollock, R. E., Weichselbaum, R. R., Holland, J. F. and Frei, E. Cancer Medicine, 5^{th} edition, B. C. Decker, 2000.
2. Levin, V. Chemotherapy for Brain Tumors of Astrocytic and Oligodendroglial Lineage. Neuro-oncol. 1, 69-80, 1999.
3. Struck, R. F., Kirk, M. C., Witt, M. H., Laster, W. R. Isolation and Mass Spectral Identification of Blood Metabolites of Cyclophosphamide. Biomed. Mass Spectrom 2, 46-52, 1975.
4. Benckhuijsen, C., Van Der Steen, J. and Westra, J. G. Vortrag VII, International Chemotherapy Kongress, Prague, 1971, B-3/9.
5. Struck, R. F., Thorpe, M. C., Coburn, W. C. and Laster, W. R. Cyclophosphamide. Complete Inhibition of Murine Leukemia L1210 In Vivo by a Fenton Oxidation Product. J. Amer. Chem. Soc. 96, 313-315, 1974.
6. Montgomery, J. A. and Struck, R. F. Synthesis and Structure-Activity Relationships of Pre-Activated Analogs of Cyclophosphamide. Cancer Treat. Rep. 60, 381-393, 1976.
7. Fuchs, H. E., Archer, G. R., Colvin, O. M., Bigner, S. H., Schuster, J. M., Fuller, G. N., Muhlbaier, L. H., Schold, S. C., Jr., Friedman, H. S. and Bigner, D. D. Activity of Intrathecal 4-Hydroperoxycyclophosphamide In A Nude Rat Model of Human Neoplastic Meningitis. Cancer Res. 50, 1954-1959, 1990.
8. Schuster, J. M., Friedman, H. S., Archer, G. E., Fuchs, H. E., McLendon, R. E., Colvin, O. M. and Bigner, D. D. Intraarterial Therapy of Human Glioma Xenografts In Athymic Rats Using 4-Hydroperoxycyclophosphamide. Cancer Res. 53, 2338-2342, 1993.
9. Colvin, M. and Hilton, J. Pharmacology of Cyclophosphamide and Metabolites. Cancer Treat. Rep. 65, Suppl. 3, 89-95, 1981.
10. Ojwang, J. O., Grueneberg, D. A. and Loechler, E. L. Synthesis of a Duplex Oligonucleotide Containing a Nitrogen Mustard Interstrand DNA-DNA Cross-link. Cancer Res. 49, 6529-6537, 1989.
11. Millard, J. J., Raucher, S, and Hopkins, P. B. Mechlorethamine Cross-links Deoxyguanosine Residues at 5′-GNC Sequences in Duplex DNA Fragments. J. Amer. Chem. Soc. 112, 2459-2460, 1989.
12. Bauer, G. B. and Povirk, L. F. Specificity and Kinetics of Interstrand and Intrastrand Bifunctional Alkylation by Nitrogen Mustards at a G-G-C Sequence. Nucleic Acids Res. 25, 1211-1218, 1997.
13. Dong, Q., Barsky, D., Colvin, M. E., Melius, C. F., Ludeman, S. M., Moravek, J. F., Colvin, O. M., Bigner, D. D., Modrich, P. and Friedman, H. S. Formation of a Single Phosphoramide Mustard-induced DNA Interstrand Cross-link in an Oligonucleotide Duplex. Proc. Natl. Assoc. Sci. USA 92, 12170-12174, 1995.
14. Struck, R. F., Davis, R. L., Berardini, M. D.; Loechler, E. L. DNA Guanine-Guanine Crosslinking Sequence Specificity of Isophosphoramide Mustard, the Alkylating Metabolite of the Clinical Antitumor Agent Ifosfamide. Cancer Chemother. Pharmacol. 45, 59-62, 2000.

### RELATED REFERENCES

1. Takamizawa, A., Matsumoto, S, and Iwata, T. 4-Hydroxycyclophosphamide anhydro-dimer: revised structure of the Fenton oxidation product of cyclophosphamide. Tetrahedron Lett. 6: 517-520, 1974.
2. Stemglanz, H., Einspahr, H. M., Bugg, C. E. Structure and conformation of 4-peroxycyclophosphamude. A cytotoxic oxidation product of cyclophosphamide. J. Am. Chem. Soc. 96:4014-4015, 1974.
3. Benckhuysen, C., van der Steen, J., Spanjersberg, E. J. Two stable Fenton oxidation products of cyclophosphamide (NSC-26271) as precursors of 4-hydroxycyclophosphamide (NSC-196562) under physiologic conditions. Cancer Treat. Rep. 60:369-372, 1976.

What is claimed is:

1. A compound represented by the formula
C₁₄H₂₈Cl₄N₄O₆P₂
2H-1,3,2-oxazaphosphorin-2-amine, 4,4′-dioxy-bis[N,N-bis(2-chloroethyl]-tetrahydro-, 2,2′-dioxide
PeroxyCPA.

2. The compound of claim 1 being peroxyCPA.

3. A pharmaceutical composition comprising the compound of claim 2 or an addition salt thereof and a pharmaceutically acceptable carrier.

4. The use of peroxyCPA in treating human cancer, particularly malignant primary brain tumors and malignant metastatic tumors in the brain, particularly breast tumors.
